(19) United States
(12) Patent Application Publication
(10) Pub. No.: US 2008/0216846 A1
(43) Pub. Date: Sep. 11, 2008

(54) SPINAL INTERVENTION TECHNIQUES AND INSTRUMENTS FOR POST-LAMINECTOMY SYNDROME AND OTHER SPINAL DISORDERS

(76) Inventor: Bruce LEVIN, Philadelphia, PA (US)

Correspondence Address: DUANE MORRIS, LLP;IP DEPARTMENT, 30 SOUTH 17TH STREET, PHILADELPHIA, PA, 19103-4196, US

(21) Appl. No.: 12/029,174

(22) Filed: Feb. 11, 2008

Related U.S. Application Data
(63) Continuation of application No. PCT/US2006/031172, filed on Aug. 10, 2005.
(60) Provisional application No. 60/707,297, filed on Aug. 10, 2005.

(51) Int. Cl. A61B 19/00

(52) U.S. Cl. 128/898

(57) ABSTRACT

The invention relates to methods and instruments for relieving spinal nerve impingement disorders (SNIDs) and symptoms associated with SNIDs. The methods involve separating a spinal neural structure and a transforaminal or peliforaminal ligament.

### CROSS-REFERENCES TO RELATED APPLICATIONS

This application is a continuation in Part of co-pending U.S. Application No. PCT/US2006/031172 filed 10 Aug. 2006, which is now (abandoned).

### BACKGROUND OF THE INVENTION

The invention relates generally to the field of surgical techniques and instruments.

It is known that impingement of non-neural tissues on spinal neural structures (SNSs, including both the spinal cord and the spinal nerves) can produce symptoms such as pain, numbness, and muscle weakness in body areas including or innervated by the impinged nerve. The physical location of nerve impingement can be distinct from the body location at which a symptom of the impingement is perceived. It can therefore be difficult to correlate symptomology with causation. As a result, many surgical interventions intended to relieve the symptoms fail. Furthermore, surgical interventions, whether successful or not, can result in development of scar tissue, fibroid adhesions, or other non-neural tissue structures that can impinge on SNSs, thereby complicating or worsening the disease state and its symptoms.

It is known that herniated or bulging intervertebral discs can impinge on a spinal nerve root, resulting in radicular pain, sciatica, arm pain, or other symptoms, depending on the physiological location on the nerve root impingement. Impairment of sensory and motor nerve functions are also known symptoms of spinal nerve impingement.

Epidural steroid injections, for example, have met with varying rates of success depending upon disease entity, patient, intercuitent social, economic, and psychological issues, and mode of delivery. Success rates of surgical interventions vary according to these and other variables as well. In general, further deterioration and scar formation limit long term outcome successes in many patients.

The anatomy of spinal neuroforamina is understood, and it is known that anatomical differences among individuals are common, as are anatomical changes in a single individual over time or as a result of physical and other stresses exerted upon an individual's body.

There remains a need to improve the efficacy and safety of interventional surgical techniques to optimize patient outcome. The present invention satisfies this need.

### BRIEF SUMMARY OF THE INVENTION

The invention relates to methods and instruments for relieving a spinal nerve impingement disorder (SNID) of a vertebrate such as a human. The methods comprise percutaneously inserting an instrument into a spinal neuroforamen, or very near (e.g., within 1-10 millimeters of) the external opening of a spinal neuroforamen, and displacing a non-neural tissue from the spinal neural structure (SNS) sufficiently to relieve the disorder. The non-neural, non-intervertebral-disc tissue can be moved within, expelled from, cut, lysed, stretched, ablated, or removed from the neuroforamen or from the SNS. Regardless of the method, the geometric, spatial, pressure, stress, or strain relationship of the non-neural tissue and the SNS is altered sufficiently to relieve the disorder.

The invention also relates to methods and instruments for assessing the location of impingement associated with a SNID of a vertebrate. The methods comprise stimulating the body of the vertebrate at a plurality of physical locations innervated by different portions of the SNS and assessing the neuronal response to each stimulus. The location of compression can be assessed by observing decreased response by portions of the SNS distal to the location.

### DETAILED DESCRIPTION OF THE INVENTION

The invention relates to methods and instruments for relieving spinal nerve impingement disorders (SNIDs) and symptoms associated with SNIDs. The methods involve inserting an instrument into a spinal neuroforamen (preferably percutaneously) to separate a spinal neural structure (SNS) and a non-neural tissue. In another embodiment, the instrument is used to separate the SNS from a non-neural tissue that occurs at or near (within 1 centimeter, and more likely with 5 or 3 millimeters of) the lateral opening of an intervertebral foramen. In this lateral embodiment, separation of the SNS from a ligament, false ligament, or other collagenous fibers can relieve stress on the SNS, resulting in relief of symptoms of nerve impingement.

The methods described herein have been demonstrated to provide substantial relief to human patients who were afflicted with painful SNIDs and who did not respond well to known treatment methods.

The invention further relates to methods of assessing the physical location of SNS impingement. These methods can be used in conjunction with the methods described herein or with known surgical or therapeutic methods to provide relief to subjects afflicted with a SNID.

Definitions

As used herein, each of the following terms has the meaning associated with it in this section.

A “spinal neural structure” (“SNS”) is a nerve, a branch of a nerve, a bundle of nerves, or an individual neuron that is present within the vertebral column of a vertebrate along at least a portion of the origin or anatomic course of the nerve, branch, bundle, or neuron. As used herein, at least the spinal cord and the 31 pairs of spinal nerves of a human are included within the scope of the term “spinal neural structure.” SNSs include sympathetic and parasympathetic nerves present within or associated with tissues of the vertebral column of a vertebrate. Disruption or freeing of these structures may improve blood flow to related radicular neural structures and thereby improve their physiologic functions and decrease symptoms. SNSs can also include pain sensitive neuronal structures that may mediate pain in normal or diseased discs and related structures as well as those that transmit pain in disc, vertebral body or endplate structures.

A “spinal nerve impingement disorder” (“SNID”) is an abnormal physiologic or perceived condition of a vertebrate associated with one or more of contact of an SNS of the vertebrate with a non-neuronal tissue, compression of an SNS of the vertebrate by a non-neuronal tissue, and adhesion of a non-neuronal tissue to an SNS of the vertebrate. Known examples of SNIDs include bulging and herniated discs, spondyloses, spondylolistheses (slipped discs), and post-laminectomy syndrome (e.g., those associated with fibroid adhesions to spinal nerve roots or related SNSs or by scar tissue-induced alteration of normal SNS anatomy, physiology, or function).

A disorder is “relieved” if the severity of the disorder or of a symptom thereof is lessened. Reduction or elimination of pain, numbness, or muscle weakness symptomatic of a SNID are all examples of relief of the SNID.

A “transforaminal ligament” is a band of collagenous or other fibrous tissue that traverses at least a portion of the outer (lateral) end of an intervertebral foramen. Several transforaminal ligaments are recognized in the art, including the superior and inferior corporotransverse ligaments, the superior transforaminal ligament, the middle transforaminal ligament, and the interior transforaminal ligament, and these recognized structures are included within the scope of this term. It is furthermore recognized that some or all of these structures are absent in individual humans, the particular absent structures varying among individuals. In addition, it is recognized that other fibrous bands of tissue exist or develop transforaminally in individuals, and that not all of these structures will have established names or locations, the location, length, and thickness varying among individuals. Transforaminal ligaments need not be true ligaments; it is recognized that some transforaminal ligaments (e.g., the inferior transforaminal ligament) extend between processes of a single bone.

A “periforaminal ligament” is a band of collagenous or other fibrous tissue that contacts a SNS at or near the outer (lateral) end of an intervertebral foramen, generally within one centimeter of the external face of the foramen. Like transforaminal ligaments, periforaminal ligaments vary in location, length, and thickness among individual humans, and many periforaminal ligaments will not have recognized anatomic designations. Like transforaminal ligaments, some periforaminal ligaments are not true ligaments. The mamillo-accessory ligaments (which are not true ligaments) are examples of periforaminal ligaments.

In the context of an SNS, a first location is more distal than a second location if the first location is farther (along a neuronal path) from the spinal cord than the second location.

Description

The invention relates to a method of relieving a spinal nerve impingement disorder (SNID) of a vertebrate such as a human. The method comprises inserting an instrument into, or near the lateral opening of, a spinal neuroforamen associated with the SNID and displacing a non-neural tissue from the SNS sufficiently to relieve the disorder. The method is suitable for minimally-invasive surgical procedures, such as percutaneous insertion of one or more devices into or near the neuroforamen or insertion of the devices through one or more relatively small incisions.

By way of example, the methods described herein can include blunt or non-blunt dissection of a SNS and non-neural tissues using a single instrument. This can be performed under visualization or other observation of the instrument, the SNS, one or more non-neural tissues, or the borders of the neuroforamen to permit sensitive control over the procedure by the attending medical officer. This procedure effects a separation of neural and non-neural tissues which alters the spatial or physical relationship (mechanically or otherwise) and has been found to be surprisingly effective for relieving SNIDs and their symptoms.

An important aspect of the methods described herein is the discovery that the methods can be performed within the neuroforamen or near (e.g., within 1 centimeter of) the lateral opening thereof. Preferably, the methods are performed in conjunction with a technique whereby the inserted instrument or one or more tissues in or near the neuroforamen (or the borders of the neuroforamen itself) can be observed (e.g., visually or neurographically) contemporaneously with performance of the methods. By way of example, an SNS in the neuroforamen can be observed by a magnetic resonance, computerized tomography, fluoroscopic, ultrasound, or other method or using an endoscope or camera attached to or contained within the inserted instrument while non-neural tissue in the foramen is manipulated with the instrument inserted therein. Electrographic methods of observing a nerve are known and can be used to monitor an SNS during performance of the methods described herein. Owing to the criticality of SNS integrity, the SNS is preferably among the tissues observed or monitored.

It was previously known that impingement of a non-neural tissue (e.g., bone or intervertebral disc material) on an SNS can result in a variety of symptoms, including axial or radicular pain, weakness, numbness, and dysesthesia. Previously known therapeutic interventions are centered around delivery of therapeutic chemicals to the site of impingement or surgical relief of the impingement, such as by surgical bulk removal of impinging non-neural tissue, fusion of vertebral joints, or some combination thereof. A shortcoming common to these prior techniques is that their efficacy relies on identification of the correct location of the SNS impingement. Direction of known therapy to another location often fails to provide significant relief to the subject.

A further shortcoming of prior surgical techniques is that they are normally performed using relative larger incisions through multiple layers of tissue (e.g., skin, muscle, and spinal lamina). Significant tissue trauma results from such incisions and currently used surgical techniques and manipulations (e.g., endoscopic procedures). General anesthesia of the subject is required for such invasive surgery (even current endoscopic methods), and this precludes the patient from providing sensory or motor feedback during the procedure. Furthermore, post-surgical developments (e.g., formation of fibroid adhesions or generation of scar tissue) can complicate or aggravate the SNID, regardless of the short-term success or failure of the surgery.

Examples of normal disc pressures are as follows, for an unloaded disc 20 psi; for a disc in a standing individual, 50 psi; for a disc in a sitting individual, 90 psi; for a disc in an individual who is lifting a heavy item, 200 psi.

Chemical sensitive discs have pain at minimal pressure. 15 psi above opening pressure was chosen as the threshold for a chemical disc, as this is well below the mechanical load resulting from sitting. Mechanical discs have pain provoked at pressures between standing and lying, that is between 15 and 50 psi above opening pressure. Indeterminate discs have pain between 51 and 90 psi above opening pressure and normal discs have no pain.

Foraminal Procedure

An important aspect of the interventional methods described herein is the discovery that they can be performed with precision within the intervertebral neuroformina with a single instrument, which can be smaller than those currently in use and without the need for a second inserted device to visualize the first. As a result, the subject experiences less trauma, lower risk of scarring, and lower incidence of recurrence of SNID symptoms. By displacing non-neural tissue from an SNS in a spinal neuroforamen, SNIDs and their symptoms can be substantially relieved. When the methods described herein are performed in a minimally-invasive percutaneous manner, the trauma and post-surgical complications associated with traditional spinal surgery can be lessened or eliminated.

The manner in which the non-neural tissue is displaced from the SNS is not critical. By way of example, it can be displaced by resection of the non-neural tissue, by pushing or pulling the non-neural tissue out of the neuroforamen, or by moving the non-neural tissue within the neuroforamen to a position at which it does not as significantly impinge upon the SNS. The non-neural tissue and the SNS can be separated by manipulating either or both tissues. That is, the non-neural tissue can be manipulated in order to displace it away from the nerve. The SNS can be manipulated in order to displace it away from the non-neural tissue. Special care should be taken not to sever or damage the SNS if it is manipulated, consistent with known precautions for surgical manipulation of nerve tissue.

One or more instruments can be introduced into the neuroforamen directly by a percutaneous route or, optionally or in addition, by another route such as through a catheter positioned in the epidural space or in the intrathecal space. When vertebral disc, scar tissue, or other material is encountered, the instrument separates the non-neural material from the nerve. This can be achieved, using a rigid or resilient surgical instrument, by downward or outward deflection or traction applied using the instrument. Such deflection or traction can move the non-neural material within the neuroforamen or force or draw it out of the foramen. Alternatively, a gouging, cutting, abrading, disrupting, ablating, or other tissue-destroying instrument can be used to remove or degrade the non-neural material, thereby facilitating its removal from the neuroforamen—either by the same or a different instrument or by suction, aspiration, or irrigation. If the non-neural material is compressible, then an expandable device (e.g., an inflatable balloon or an expandable wire mesh) can be used to compress the material and limit or prevent its impingement upon the SNS. By way of example, balloon type intra-foraminal devices can be used to displace or flatten non-neural material to gain more room intra- or peri-foraminally or to free the SNS from impingement by the material. Alternatively, a deflated balloon, or a partially-collapsed or deformable device, can be inserted into a neuroforamen, inflated (or otherwise expanded in one or more geometric planes), and drawn distally to dislodge disc fragments, scar tissue, or other materials from the SNS, the neuroforamen, or both.

As will be apparent to a skilled artisan in this field, substantially any device that lessens impingement of the non-neural material upon the SNS can be used in these methods.

If the non-neural material impinging the SNS is bony or mineralized, then a specialized instrument may be required. In particular, an instrument capable of removing such material should be used. By way of example, a drilling, abrading, or cutting instrument can be used. Preferably, an instrument of this type includes a shield between the tissue cutting or abrading portion of the device and the SNS near which it will be placed. The shield can be shaped and sized to provide protection against neurotrauma. By way of example, an instrument can have a generally cylindrical cutting head having teeth on its curved surface and contained on about one half or two thirds of its circumference within a larger, smooth, generally cylindrical shield, such that the cutting head can be engaged with the non-neural material in the neuroforamen and shielded from the SNS in the neuroforamen. Design and operation of surgical devices of this type are within the ken of the ordinarily skilled worker in this field.

The identity of the instrument used to perform these procedures is not critical, and skilled artisans will recognize that a variety of known instruments can be used. Furthermore, design of instruments adapted to perform these procedures is within the ken of the ordinary surgical instrument designer. The device can, for example, be a needle, introducer, catheter, forceps, decompressor device, saw, macerator, ultrasonic, laser, or any other known device of appropriate composition and relatively small size. The device can be asymmetric along one or more of its axes. The device can be constructed of a material which is X-ray or MRI lucent or opaque, with or without radiological or MRI discernable markings. Materials used in devices suitable for MRI can be non-ferrous, or can be constructed of polymer, glass, carbon, nanotubules, or other substances to minimize scatter and distortion and to optimize viewing and to avoid magnetic issues. Instruments used in conjunction with CT should be made from material likewise selected to reduce scatter. The device preferably has one or more viewable markings to assist determination of the location and orientation of the device in the patient and relative to various tissues and anatomic landmarks. This maximizes effective execution of the procedures. In utilizing MRI or CT guidance with or without three-dimensional reconstruction, a better view of essential anatomy is offered the operator and without the inability to see around corners or other obstacles in the field of view, or have blood or other fluids obstruct direct visualization. However, the ability to perceive and control orientation of instruments relative to tissues is important. For example a cutting or ablative edge should be positioned to contact scar, disk, bone, or other tissues while sparing neural and vascular structures. In post-laminectomy syndrome for example, the cutting edge of the device may need to rotate, twist, advance, retract, and change orientation to closely follow the course of the radicular nerve to remove impinging structures throughout the course of the nerve. Similarly, a simple MRI-compatible forceps can be designed or have markings to provide orientation of the device in relation to disc fragments to maximize grasping and visualization in an open or closed position. An ultrasonic device, laser, or other device designed to disrupt disc fragments can also have markings or an asymmetric design to avoid neurotrauma.

The procedures are amenable to performance using ordinary surgical instruments, micro-machine-type (or other minimally-invasive) instruments, and robotically- or remotely-operated instruments, for example. Non-limiting examples of suitable devices include picks, needles, probes, cannulas, elevators, spatulas, spoons, separators, dissectors, hooks, awls, burnishers, rasps, curettes, drills, burrs, screws, trephines, scalpels, scissors, forceps, retractors, pliers, syringes, balloons, and the like. The instrument can also be a device adapted to deliver a tissue- or other material-destroying agent (e.g., heat, cold, electricity, abrasion, ultrasound, vibration, laser light, maser radiation, fluid pressure, gamma radiation, microwave energy, or a chemical or biochemical agent) to a selected site. Hybrid instruments that combine two or more of these functionalities are also useful in these methods. By way of example, an instrument including functional portions for grasping and grinding (or otherwise degrading) a selected tissue and also including a suction port for removing debris can be used. Multiple functionality of a single instrument is particularly advantageous for instruments to be used in or through the narrow confines of a spinal neuroforamen because they lessen the need for repositioning or replacement of instruments and the risk of trauma to neural, vascular, and other structures that can accompany instrument manipulation.

Another example of an instrument contemplated for use in the methods described herein is a shielded tissue remover adapted for use in or through a spinal neuroforamen. Such a tissue remover has a tissue-removing mechanism that is wholly or partially shielded from contacting surrounding tissues. The shielding (e.g., a sleeve enveloping all or part of the tissue-removing mechanism) serves to prevent unintended damage to tissues that lie close to a tissue to be removed or that lie along the path of insertion of the device. Upon activation by an operator, the device removes tissue selected by the operator by engaging an unshielded portion of the tissue-removing mechanism against the tissue to be removed. Such tissue removal can be effected by mechanical methods (e.g., by cutting or abrading the tissue) or by chemical or physical methods (e.g., by application of a chemical agent, heat, cold, radiation, or laser illumination). The instrument preferably is constructed such that it has a controlled imaging profile, so that it can be observed and precisely positioned by observation using an imaging method (preferably using an imaging method in which impingement upon a SNS can also be observed, so that the instrument can be positioned to alleviate the impingement). The instrument can have a shape or markings that are resolvable under imaging to facilitate appropriate positioning of the device by the operator.

Visualization

Owing to the importance of preventing SNS damage during the procedures described herein, it is preferable that those procedures be performed while observing one or more of the tissues present in or near the neuroforamen involved in the procedure. Preferably, the SNS is observed or visualized, given its importance. The devices and methods used for visualization or observation are not critical, and it is recognized that a wide variety can be suitable for use in the methods described herein.

Introduction of an instrument into the neuroforamen can be guided by any known imaging technique, such as normal or ultrafast magnetic resonance imaging, Doppler-based imaging, or computer-assisted tomography. Imaging-guided introduction can improve the effectiveness of the treatment, reduce scarring, and improve the safety margin of the procedure. Performance of a neurogram can be effective for locating the nerve root, which serves to mark the borders of the neuroforamen. Design and manufacture of instruments that are visible, invisible, or have visible marks for particular imaging techniques are known, and instruments suitable for use with substantially any imaging technique in the methods described herein can be made by a skill artisan without significant, if any, experimentation.

During the procedures described herein, the vertebrate can be partially awake (e.g., subject only to local anesthesia in the region of the procedure) so that the patient can describe perception of symptoms of the SNID (or relief thereof) to assist in avoiding neurotrauma. Similarly, neurophysiologic, or electrophysiological monitoring (e.g., somatosensory evoked potential, electromyography and nerve conduction velocity, or other neural tests) can be used to document improved neurologic functioning and to avoid unnecessary neurotrauma. As described in the examples herein, substantial relief from SNID symptoms could be perceived by human patients in the time periods during which the procedures were performed, permitting the attending physician to discontinue the procedure (and the attendant risk of neurotrauma) upon achievement of relief from symptoms.

Disorders

The methods described herein are effective for relieving substantially any SNID characterized by contact between an SNS and a non-neural tissue or material. Examples of SNIDs that can be relieved using these methods include bulging intervertebral discs, herniated intervertebral discs, slipped discs, spondylosis, post-laminectomy syndromes, failed back syndromes, and axial and radicular pain of unexplained SNS etiology. Symptoms of these SNIDs for which relief can be obtained using the methods described herein include axial pain, radicular pain, muscle weakness, numbness, dysesthesia, and other pathophysiologic manifestations of SNS disorders.

It is recognized in the art that the term “failed back syndrome” does not describe a well-delineated physiological condition. Instead, the term is applied to patients who experience continued, different, or additional SNID or somatic, non-neuropathic symptoms following traditional surgical interventions intended to relieve the SNID. Without being bound by any particular theory of operation, it is believed that some or all of the symptoms generally attributed to failed back syndrome are instead attributable to contact, compression, or adhesion of an SNS by another tissue in response to trauma inflicted during the traditional spinal intervention. By way of example, laminectomy or other spinal surgical intervention can provoke a wound-healing response that includes formation of fibrous tissue regions that can adhere to an SNS or its root or to other tissues in such a configuration that the fibrous tissue compresses an SNS. Intraforaminal manipulation of the SNS, the fibrous tissue, an anchor of the fibrous tissue, or some combination of these, can dislodge or shift the tissues in a way that discontinues impingement of the non-neural tissue upon the SNS and permits normal nerve function. It is believed that it is this discontinuation of impingement that was experienced by patients described in the examples herein as rapid and substantial relief of SNID symptoms.

Transforaminal or Periforaminal Procedure

Another surprising discovery that has been made is that many SNIDs and their symptoms do not result merely from impingement of an intervertebral disc or bone upon a SNS, but rather from impingement of a non-neural tissue other than intervertebral disc or bone upon the SNS. Such non-neural, non-disc, non-bone tissues can produce or contribute to the SNID by impingement of these tissues alone, or in combination with impingement of disc, bone, or both disc and bone tissues. It has been discovered that SNS impingement or constraint effected by transforaminal ligaments and periforaminal ligaments can contribute significantly to development and persistence of a SNID, particularly in combination with relatively minor disc or bone impingement upon the SNS that would not, by itself, be expected to result in a SNID or its symptoms. In this way, a normally non-pathologic (e.g., very small) disc enlargement can cause SNID symptoms owing to constraint of a SNS by a periforaminal or transforaminal ligament that enhances the impingement of the disc enlargement upon the SNS.

It has been discovered that lysis, stretching, or dissection of fibrous tissue within an intervertebral neuroforamen or at or near the lateral opening of the foramen can provide very rapid and profound relief from SNID symptoms, even in patients refractory to traditional therapeutic techniques (e.g., in patients who do not respond to epidural steroids). Displacement of peliforaminal and transforaminal ligaments from a SNS has led to total or near-total resolution of radiculopathic symptoms in at least several patients, as set forth in several of the examples described herein.

Discovery of the role of periforaminal and transforaminal ligaments in the pathology of SNIDs is a significant development that enables treatment of many types of back, arm, leg, and other radicular pain that was previously poorly understood or completely not understood. In view of the teaching provided herein, skilled artisans in this field are able to use the methods described herein, alone or in combination with known procedures for relieving radicular symptoms, to treat previously refractory patients.

In the transforaminal and periforaminal procedures described herein, an instrument (blunt or non-blunt) is used to displace a non-neural tissue from a SNS at or near the lateral opening of an intervertebral neuroforamen. The manner in which the non-neural tissue is displaced from the SNS is not critical. By way of example, it can be displaced by resection of the non-neural tissue, by pushing or pulling the non-neural tissue away from the SNS or away from the neuroforamen, or by stretching or moving the non-neural tissue to a position at which it does not as significantly impinge upon the SNS. The non-neural tissue and the SNS can be separated by manipulating either or both tissues. That is, the non-neural tissue can be manipulated in order to displace it away from the SNS. The SNS can be manipulated in order to displace it away from the non-neural tissue. Special care should be taken not to sever or damage the SNS if it is manipulated, consistent with known precautions for surgical manipulation of nerve tissue.

As in the intraforaminal procedure, one or more instruments can be introduced near the neuroforamen directly by a percutaneous route or, optionally or in addition, by another route such as through a catheter positioned in the epidural space or in the intrathecal space or through a traditional spinal surgical incision. When a transforaminal or periforaminal ligament or other fibrous tissue impinging upon a SNS is encountered, the instrument separates the non-neural material from the SNS. This can be achieved, using a rigid or resilient surgical instrument, by downward or outward deflection or traction applied using the instrument. Such deflection or traction can move the non-neural material away from or around the neuroforamen. Alternatively, a gouging, cutting, abrading, disrupting, ablating, or other tissue-destroying instrument can be used to remove or degrade the non-neural material, thereby facilitating its removal from the foraminal area—either by the same or a different instrument or by suction, aspiration, or irrigation. If the non-neural material is compressible, then an expandable device (e.g., an inflatable balloon or an expandable wire mesh) can be used to compress the material and limit or prevent its impingement upon the SNS. By way of example, balloon type devices can be used to displace or flatten non-neural material to gain more room periforaminally or to free the SNS from impingement by the material. Alternatively, a deflated balloon, or a partially-collapsed or deformable device, can be inserted into a neuroforamen, inflated (or otherwise expanded in one or more geometric planes), and drawn distally to dislodge disc fragments, scar tissue, or other materials from the SNS, the neuroforamen or its lateral opening, or some combination of these.

As will be apparent to a skilled artisan in this field, substantially any device that lessens impingement of the non-neural material upon the SNS can be used in these methods.

If the non-neural material impinging the SNS is bony or mineralized (e.g., a calcified transforaminal or periforaminal ligament), then a specialized instrument may be required. In particular, an instrument capable of removing such material should be used. By way of example, a drilling, abrading, or cutting instrument can be used. Preferably, an instrument of this type includes a shield between the tissue cutting or abrading portion of the device and the SNS near which it will be placed. The shield can be shaped and sized to provide protection against neurotrauma. By way of example, an instrument can have a generally cylindrical cutting head having teeth on its curved surface and contained on about one half or two thirds of its circumference within a larger, smooth, generally cylindrical shield, such that the cutting head can be engaged with the non-neural material in the neuroforamen and shielded from the SNS in the neuroforamen. Design and operation of surgical devices of this type are within the ken of the ordinarily skilled worker in this field.

The identity of the instrument used to perform these procedures is not critical, and skilled artisans will recognize that a variety of known instruments can be used. Furthermore, design of instruments adapted to perform these procedures is within the ken of the ordinary surgical instrument designer. The instrument can, for example, be any of those described herein in connection with the intraforaminal procedure.

As with the intraforaminal procedure described herein, it is important to prevent SNS damage during the transforaminal and periforaminal procedures described herein, and the same or analogous observational and precautionary procedures can be used.

The transforaminal and periforaminal procedures described herein are effective for relieving substantially any SNID characterized by extraforaminal contact between an SNS and a non-neural tissue or material. Examples of SNIDs that can be relieved using these methods include bulging intervertebral discs, herniated intervertebral discs, slipped discs, spondylosis, post-laminectomy syndromes, failed back syndromes, and axial and radicular pain of unexplained SNS etiology. Symptoms of these SNIDs for which relief can be obtained using the methods described herein include axial pain, radicular pain, muscle weakness, numbness, dysesthesia, and other pathophysiologic manifestations of SNS disorders.

Combination Methods

The intraforaminal, transforaminal, and periforaminal procedures described herein can be used alone or in combination with previously known procedures for alleviating SNIDs. By way of example, numerous percutaneous and traditional surgical discectomy procedures are known to be effective for reducing symptoms of SNIDs in which a bulging or herniated disc causes or contributes to the symptoms. SNIDs can result from multiple causes, such as a combination of a bulging disk and constraint or compression of a SNS by a transforaminal ligament, constraint or compression of a SNS by a periforaminal ligament, constraint or compression of a SNS by a non-neural intraforaminal tissue, or some combination of these. It is recognized that performance of multiple procedures can provide greater relief of SNID symptoms than performance of a single procedure, especially in patients afflicted with SNIDs having multiple causes.

By way of example, a relatively minor intervertebral disc bulge can impinge upon a spinal nerve root and urge it laterally through the neuroforamen to a small degree that would normally not result in SNID symptoms. However, if the root is adhered to (or otherwise constrained by) a periforaminal ligament, compression of the root between the bulge and the ligament can induce SNID symptoms of no obvious etiology. In such a patient, percutaneous discectomy alone (e.g., performed using a device such as the DEKOMPRESSOR (RTM, Stryker Corporation) percutaneous discectomy probe) may provide limited relief of SNID symptoms. Similarly, performance of only the periforaminal procedure described herein may also provide limited relief of SNID symptoms. Performance of both procedures may provide greater relief of the symptoms.

In patients in whom disc enlargement, bulging, or herniation causes impingement upon a SNS, it has been recognized by others that surgical removal of a portion of the disc material can relieve the impingement. The present inventor recognized that the portion of the disc can be removed from the bulk matrix of the disc (as in the prior art) or from a portion of the disc that constitutes or is very near (e.g., within 1 centimeter of) the portion of the disc that constitutes the bulge or hernia. This material can be removed by a probe inserted into or incision on the same side of the disc as the bulge or hernia. Alternatively, a probe (e.g., one such as the DEKOMPRESSOR (RTM) probe) can be inserted into another side (e.g., approximately the opposite side, or through a foramen other than the foramen of the impinged-upon SNS) of the disc, and a portion of the disc that constitutes or is very near (e.g., within 1 centimeter of) the portion of the disc that constitutes the bulge or hernia can be removed. As another alternative, the bulging or herniated portion of the disc can be secured using an anchor (e.g., a surgical suture) that is fixed to another portion of the patient's anatomy, such as the opposite side of the disk (using a suture extending within the disc) or a portion of a vertebra adjacent the disc.

Diagnostic Methods

Many patients afflicted with a SNID are evaluated with a heavy reliance on radiological findings, MRI findings, or both. These can be overstressed as primary diagnostic modalities as opposed to patient history and subtle clues found on physical examination. For example, many patients may have true radicular complaints by history with corresponding physical findings which may not have reached classic stages and have MRI findings which document such findings as very minimal disc protrusion, or very mild disc bulging, discs which contact but do not compress nerve roots, or which only touch the thecal sac but do not cause cord compression or deformity.

The degree of mechanical contact of a disc bulge, herniation, or arthritic joint component as assessed by imaging studies is often physiologically grossly underestimated. This difficulty is compounded by often variable and irreproducible electrophysiological studies which frequently miss more central neural component involvement in favor of more peripheral etiologies. When symptoms, history, and physical exam correlate with often minor findings on imaging studies in terms of neurologic distribution, these minor findings can represent the anatomic foci of severe pathophysiology processes. Furthermore, if there are significant imaging findings in remote locations, this can be a marker of physiologic trespass at other spinal locations. This is often the case with cervicogenic headaches.

Although a cervicogenic headache may originate at the C2-3 spinal levels in a human, a herniated disc or arthritic changes at C7 level may be a marker that gravitational, stress, or other mechanical factors have effected physiologic trespass along the entire C spine, even though imaging studies are not strongly positive at C2 or C3. This may be likened to a train crash where the first few cars are severely damaged, while on gross appearance the last cars are not. However, detailed inspection of these cars on the inside would reveal some damage that is not very noticeable on cursory evaluation.

The invention includes a more effective modality of evaluation based primarily on patient complaints history, physical exam, and other circumstance wherein imaging studies are used merely as clues. This aspect of the invention relates to a method of assessing which SNS is being impinged in a vertebrate afflicted with a SNID. The method comprising stimulating a portion of the body of the vertebrate at a plurality of physical locations innervated by different SNSs in order to assess which SNS is involved in the SNID. Once an involved SNS is identified, different portions of the SNS can be stimulated to identify the approximate position of the impingement. The SNS and its impinged portion can be identified by assessing the neuronal response to the applied stimuli. SNSs and portions thereof that are affected by impingement exhibit less (or no) response to stimuli distal to the point or region of impingement.

By way of example, the invention includes a method of stimulating different areas within different neuroforamina to determine which level or levels and which locations within a neuroforamen are most clinically relevant. The invention also includes improvements of commonly utilized provacative discography, A technique in which needles are inserted into one or more vertebral discs of a patient. Injection of a fluid (e.g., saline or a contrast agent) into the interior of the disc tends to increase disc volume and may provoke pain in individuals who have disc pathology and who are suffering from discogenic axial pain or pain of a radicular nature (e.g., by virtue of its impingement upon nearby SNSs.) Concordant symptoms and their intensity and degree of similarity to a patients complaints indicate that that disc may be a pain or symptom generator for that patient. The intradisc pressure and or volume of injectate at which symptoms are first described are noted. In general, normal discs will produce pain or pressure symptoms at maximal pressures or volumes, but only diseased discs will become symptomatic at midrange pressures or volumes. Symptoms at low pressures or volumes may be false positive discs, i.e., in patients with psychological amplification of symptoms, or is unsure of what he is sensing, or may indicate severe pathology or a chemically sensitive disc. There is significant patient variability as to the accuracy of discographies, and many patients have undergone surgical discectomies at the wrong level, or have had altogether unnecessary surgery on discs. Hence, there is a need for improved accuracy as to whether a patient has pain that is disc related and to better isolate symptomatic discs. One source of error is that a patient may be unsure as what he is feeling during discography and may amplify or exaggerate normal pressure and related sensations to reflect concordant symptoms therefore the invention includes two major improvements. During discography, a patient is aware that discography is taking place at a single level unknown to the patient. After that level is done, a different level is pressurized and the response noted. Hence the test is done in series, one by one. For example, L45 can be pressurized first, then L34, then L5S1, and lastly L23 or L12 as a control. The resultant symptoms and pressures are tabulated, and the level or levels which reproduce the patient's symptoms most intensely and concordantly are noted. These discs may then be operated upon. There is currently no teaching whatsoever to attempt to reaspirate injected saline or contrast. It should be noted that withdrawal of the fluid previously injected into a disc tends to decrease its volume. Should a patient note possibly concordant symptoms at a given pressure or volume of injectate, these have been demonstrated by the inventor to decrease at a certain pressure or volume and disappear entirely at a further decrease in pressure or volume. Hence, aspirating injectate will increase the accuracy of the evaluation at a given disc level. Furthermore, this enables one or more repetitions of re-injecting said saline or contrast into the same disc at the same or another time during the procedure should the patient ascribe concordant symptomatology at similar pressures or volumes, that level can truly be said to be a symptom generator. Furthermore, by utilizing a manifold where all studied discs can be randomly injected or aspirated at points in time identical or varied, accuracy can be improved. By repeatedly injecting a fluid into a patient's disc and withdrawing the fluid therefrom, the patient is enabled to describe the resulting sensations. Comparison of the patient's sensational reactions to these manipulations and the patient's symptoms can be made. Close correspondence (e.g., degree and location of pain) between the sensations and the symptoms indicates which manipulated discs are involved in the patient's symptomology and that treatment of those discs, as described herein, is likely to benefit the patient's symptoms and/or the underlying disorder. Similarly, when multiple discs are candidates for involvement in the patient's symptoms, this injection/withdrawal technique can be used to probe the involvement of each of the discs, either serially or in parallel (e.g., using a fluid injection/withdrawal manifold system operably connected with multiple needles inserted into the candidate discs). Using these techniques, the inventor has had better identification of which discs have been symptomatic, and percutaneous discectomies have been much more effective.

By way of example, the invention includes a method of stimulating different areas within different neuroforamina to determine which level or levels and which locations within a neuroforamen are most clinically relevant. The invention also includes a diagnostic discography technique in which needles are inserted into one or more vertebral discs of a patient. Injection of a fluid (e.g., saline or a contrast agent) into the interior of the disc tends to increase its volume and its impingement upon nearby SNSs. Conversely, withdrawal of the fluid previously injected into a disc tends to decrease its volume and its impingement upon nearby SNSs. By repeatedly injecting a fluid into a patient's disc and withdrawing the fluid therefrom, the patient is enabled to describe the resulting sensations. Comparison of the patient's sensational reactions to these manipulations and the patient's symptoms can be made. Close correspondence (e.g., degree and location of pain) between the sensations and the symptoms indicates that manipulated disc is involved in the patient's symptomology and that treatment of that disc, as described herein, is likely to benefit the patient's symptoms and/or the underlying disorder. Similarly, when multiple discs are candidates for involvement in the patient's symptoms, this injection/withdrawal technique can be used to probe the involvement of each of the discs, either serially or in parallel (e.g., using a fluid injection/withdrawal manifold system operably connected with multiple needles inserted into the candidate discs).

Determination of the symptomatic nerve segment(s) can be made using the methods described herein in situations in which imaging or other studies do not fit optimally with clinical picture utilizing foraminal or nerve root challenge,. An example of how this can be achieved follows.

A needle, cannula, or other delivery device is inserted into each of the suspected nerve roots or foraminal zones. Each foramen is challenged by introduction of contrast media, a chemical, or another fluid, or by application of electric, light, sound, ultrasound, radio, radiofrequency, magnetic, heat or microwave energy stimulation. This challenge can be performed with or without electrophysiological readings. By obtaining concordant complaint(s) from the patient for each such challenge, the likely spinal level of pain and or other symptom generation can be diagnosed.

Compressed or initated nerve roots are not equally affected at all points along the nerve root or course of the nerve. For example, a disc herniation may chemically or mechanically compromise a radicular or other nerve at only one section and in one location along that section (e.g., inferiorly). Desirable effects of medication or intervention are enhanced by placement or intervention along the location of greatest trespass. The precise location of greatest trespass is located by challenging the nerve as above in different locations along the nerve, (e.g., along three orthogonal axes about the nerve). Therapeutic interventions are directed towards those levels which are most concordant, and they may administered in series or in parallel.

Using any of the imaging techniques described herein (or, less preferably, without the aid of imaging), a trocar, endoscope, catheter, steerable catheter, grasping or dissecting mechanism, an emitter of laser or other light, ultrasound, microwave, radiofrequency, heat, or cold, or another nerve challenging device can be inserted percutaneously and used to challenge a nerve root or a nerve along its course. The nerve challenging device can, for example, be a needle or introducer, and can be asymmetric along one or more of its axes. The device can be constructed of a material which is X-ray lucent or opaque, with or without radiological marking. Materials used in devices suitable for MRI can be non-ferrous, or can be constructed of polymer, glass, carbon, nanotubules, or other substances to minimize scatter and distortion, and to optimize viewing and to avoid magnetic issues. Instruments used in conjunction with CT should be made from material likewise selected to reduce scatter. The device preferably has one or more viewable markings to assist determination of the location and orientation of the device in the patient.

Pharmaceutical Treatment

A variety of pharmaceutically active agents are known to be effective for treatment of SNIDs. However, such an agent can exhibit efficacy only if administered to a location and in such a manner that the agent is able to exert its pharmacological effect on a relevant biological structure—that is, a tissue involved in the SNID. As described herein, prior methods of treating SNIDs are hampered by difficulty identifying relevant SNSs and the location of SNS impingement.

The diagnostic methods described herein for identifying an SNS involved in a SNID and for identifying the location of an impingement on that SNS can be used to direct administration of known SNID-relieving agents to body locations at which the agents can be effective. The invention includes methods of treatment comprising epidural injection of steroid, anti-neuropathic, and anti-inflammatory compounds alone or in combination for back pain, disc disease, facet disease, spondylosis, failed back neck syndrome, radiculopathy, radiculitis, radicular symptoms, spinal stenosis, headache, migraine, cluster headache, and related disorders, including the SNIDs described herein.

Treatment can be effected by delivery of agents alone or by administration of depot formulations. Depot formulations can be made using micelles or liposomes, which can be composed or formulated with anti-inflammatory lipids or fatty acids (e.g., cetyl myristoleate) or with standard compounds, such as polyethylene glycol or related compounds, and/or other membrane stabilizing agents. Such formulations are known in the art

Examples of such agents which can be thus administered include anti-neuropathic agents, including all anti-seizure medications, such as KEPPRA®, LAMICATAL®, TOPAMAX®, TIAGABINE®, TRILEPTAL®, ZONEGRAN®, TEGRETOL®, DILANTIN®, DEPAKOTE®, NEURONTIN®, CYMBALTA®, GABATRIL®, pregabalin, carbemazapine, oxcarbazine, tricyclic antidepressants, serotonin inhibitors, ketamine and other NMDA antagonists, NSAIDs, COX2 inhibitors, calcium, sodium, potassium, chloride inhibitors, depot local anesthetics, and polyethylene glycol.

Other examples of agents which can be thus administered include anti-inflammatory agents, including TNF antagonists such as etanercept (ENBREL®, Immunex Corporation); infliximab (REMICADE®, Johnson and Johnson); D2E7, a human anti-TNF monoclonal antibody (Knoll Pharmaceuticals, Abbott Laboratories); CDP 571 (a humanized anti-TNF IgG4 antibody); CDP 870 (an anti-TNF alpha humanized monoclonal antibody fragment), both from Celltech; soluble TNF receptor Type I (Amgen); pegylated soluble TNF receptor Type I (PEGs TNF-R1) (Amgen); and a molecule containing at least one soluble TNF receptor.

Such agents can also include antagonists of one or more of the following: interleukin-1 (IL-1), IL-6, TNF-alpha, TGF-Beta; agonists of one or more of the following: IL-4, IL-10, and IL-13 agonists; and antagonists of one or more of the following: LIF, IFN-gamma, OSM, CNTF, TGF-beta, GM-CSF, IL-11, IL-12, IL-17, IL-18, IL-8 tachylcinins, VIP (vasoactive intestinal peptide), and VPF (vascular permeability factor), caspase-1, caspase-5, PYCARD, NALP1, the SIS family of cytokines, the SIG family of cytokines, the SCY family of cytokines, the platelet factor-4 superfamily of intercrines, and prostaglandins. All Dosing units are as per standard dosing regimens. The agent can also be a CPLA2 inhibitor, for example.

Pressure characteristics of lesser compliance suggest spinal stenosis or local compression by scar, disc or other material. Such characteristics can also be used to direct administration.

A transforaminal administration approach can also be used, with X-ray, fluoroscopic, ultrasound, CT scanning, MMI, or fast MRI methods used to guide administration, with or without contrast.

Expandable Artificial Intervertebral Disc Matrices

Compression of nerves or nerve roots can sometimes be alleviated by increasing separation of bones (e.g., vertebrae) impinging on the nerve or its root. Surgical removal or impinging bone (e.g., laminectomy) is known. However, methods of relieving bone or bone-induced impingement on nerves have been discovered.

In one embodiment, insertion of a swellable matrix between bones (or between different processes of a single bone) and subsequent swelling of the matrix can force the bones (or processes) apart, relieving the impingement. The matrix can be made of a material that remains in a compact state during insertion or installation and thereafter expands. The expansion can be induced by absorption of water, reaction of the matrix with water, absorption of an applied solvent, reaction of the matrix with an applied solvent, application of heat to the matrix, or some combination of these, for example. Numerous materials exhibiting such swelling properties, and selection of a swellable material suitable for residence in a human body for a period of time (e.g., hours, days, months, or years) effective to relieve a disorder associated with bone or bone-induced nerve impingement is within the level of skill of an ordinary practitioner in this field.

In another embodiment, a device which can be made to expand along an axis extending between two bones or two bone processes can be inserted therein and expanded. By way of example, the device can be a cylindrical device (e.g., having a diameter of about 1 centimeter) in which separation of the circular heads of the device can be increased by rotation of a threaded screw which extends into the device from the side of the device. Such a device can be inserted and expanded to relieve bone or bone-induced impingement on a nerve or its root. Alternatively, the heads of the device can be expanded along the axis extending between the bones or processes by injecting material (e.g., an oil, water, air or other fluid) into an expandable chamber encased within the device, thereby urging the heads outwardly from the central portion of the device. The device can exhibit resilience along the axis between the bones or processes, thereby enhancing comfort and providing a more natural feel to the affected body area. By way of example, a device having two opposed flat faces having a gas-filled bladder therebetween can be inserted intervertebrally. One or both faces of the device can be attached to, or adapted for ingrowth of bone matrix from, a bone against which the face is opposed. Design and fabrication of such devices are within the level of ordinary skill for a practitioner in this field, in view of the guidance provided herein.

Furthermore, a variety of intervertebral disc replacement devices are known and described in the literature. Substantially any of these devices can be used as described herein. Selection of an appropriate physiological location at which to implant such a device can be performed using the diagnostic methods described herein.

### EXAMPLES

The invention is now described with reference to the following Examples. These Examples are provided for the purpose of illustration only, and the invention is not limited to these Examples, but rather encompasses all variations which are evident as a result of the teaching provided herein.

### Example 1

Relief of Lumbar Back Pain

A woman with failed back syndrome did not respond to a prior trans-foraminal epidural steroid injection at L5S1 although a concordant neurogram was obtained. The next time, the needle was directed to the inferior portion of the same nerve root and challenge was more concordant. Injection of steroid at this locus was very effective in relieving her radicular symptoms.

It is also notable that in contrast to common belief, neural challenge occasionally reveals that symptoms are related to a different nerve root than is suggested by dermatomal charts. For example, a patient with radiculopathy and a large disc at L45 had symptoms of thigh pain usually referable to L34, but challenge of L45 revealed concordance at this level and not at the level of L34. The patient responded to L45 injections but not to injections at L34.

In another instance, a male patient had knee symptoms with a significant L45 disc, but had symptoms that were actually from a very minimal degree of foraminal stenosis on the same side at L34, which was more concordant on challenge that L45.

### Example 2

Herniated or Bulging disc Into the Neuroforamen

A middle aged woman had suffered from left-greater-that-right radicular symptoms including pain, numbness and weakness for over 2 years secondary to a disc bulge. She had only 10-15% relief from a targeted transforaminal epidural steroid injection at the appropriate level. During that procedure, disc material was felt to be in foramen on needle placement. Therefore, the patient was taken to the procedure suite 2 weeks later.

Usual technique was followed. A 22 gauge spinal needle was placed intra-foraminally and a neurogram was obtained. A 17 gauge Tuohey needle was then introduced at the inferior portion of the neuroforamen according to the neurogram. With its bevel away from the nerve root, the disc was distracted out of the neuroforamen accompanied by a peeling sensation noted by the physician. Concurrent with the peeling sensation the patient noted that her sensation was returning to normal and her pain had decreased significantly. At that time her motor strength returned. Steroids were administered in the usual fashion, and the patients symptoms were 95% improved on that side.

### Example 3

Post-Laminectomy Syndrome

A middle aged woman with debilitating sciatica following back surgery did not respond to transforaminal epidural steroid injections. MRI showed mild scar tissue encasing the L5 nerve root. The usual transforaminal neurogram was obtained with pressure noted on injection attempt and little spread noted. The needle was advanced in several planes to shave the scar tissue from the nerve. Slight pressure was the applied to the syringe with the patient monitored for neurotrauma symptoms. Pressure was increased and a breakthrough loss of resistance noted, followed by an improved neurogram with good spread. The patient noted her symptoms improved 80%.

Follow up MRI one week later documented a significant decrease in perineural scarring and the neuroforamen was now much more patent. The procedure was repeated this time using a Tuohey needle to scrape the foramen and her symptoms of residual buttock pain disappeared. She has done well up to several months follow up. Hence this technique represents a significant alternative to risky repeat surgery. The same technique was used to decompress scar tissue with areas of cystic changes in another post-laminectomy patient and in patients with fact cysts.

### Example 4

Spinal Neuroforaminoplasty

A 60 year old man had previously undergone back surgery and continued to be afflicted with from severe axial back pain. He was deemed a poor surgical candidate, and was declined for further procedures by several neurosurgeons. The patient required high dose narcotic medication, but this reduced pain only minimally.

The patient was assessed and it was determined his symptoms were predominantly attributable to the spinal nerve of the left L45 neuroforamen. The patient was taken to the operating room and, under fluoroscopic guidance, a #17 gauge Tuohey type needle was introduced into the lateral aspect of that foramen. Foraminoplasty was performed by blunt dissection of non-neural tissue from the nerve in the foramen, and the patient noted a decrease in his pain. The patient left experiencing little pain.

The patient returned after one week with pain located lower down on the same side which was perceived as about 50% as intense as his earlier pain had been. The patient's impression was that this pain had been present earlier, but had likely been masked by the earlier more severe pain. Because his MRI showed some scar tissue in the lower L5S1 ipsilateral neuroforamen, a similar procedure was done after two weeks at that level. The patient's perception of pain decreased by about 90% and he remained well during follow up.

### Example 5

A middle-aged woman had undergone two prior laminectomies and was treated with transforaminal epidural injections on her left side. She did well until she was involved in a motor vehicle accident. Thereafter, she developed severe leg pain and low back pain and soon experienced numbness in her leg and weakness, both of which progressed to a very significant level. The patient soon developed severe radiating leg pain and allodynia which persisted. She underwent discography but was perceived to be a poor surgical candidate.

The patient underwent the foramenoplasty procedure at the L45 and L5S1 levels on the left side. Inserting the #17 Tuohey needle into her neuroforamina was accompanied by a surprising and immediate decrease in her pain and numbness, and a remarkable normalization of her motor strength. She did well during follow up.

### Example 6

A middle-aged woman presented with severe buttock and lower back pain that had been chronic for more than two years and was not amenable to physical therapy, transforaminal steroid injection, epidural steroid injection, or other procedures. She had a light subparticular disk protrusion, but symptoms were entirely left-sided. She had a disk space calcification at L45 and no fusion or obviously significant bulging. She underwent a left L5S1 foraminoplasty (i.e., the intraforaminal procedure described herein). During the procedure, ligamentous non-disk tissue was bluntly dissected and displaced from the neuroforamen. There was never any disk uptake of contrast dye, and therefore no disk material was present in this part of the neuroforamen.

Following distraction of the ligamentous structure from the neuroforamen, the patient noticed a sudden and profound decrease in her pain which has remained permanent for several months. She had not had any benefit from prior procedures. This Example demonstrates that the ligamentous structure encountered contributed to her pathology.

### Example 7

A 35-year-old man presented with severe right back and buttock pain associated with foot and leg numbness on the right side. His magnetic resonance imaging results showed a diffuse disk bulge and a right paracentral disk extrusion producing severe right lateral recessed stenosis. He had a reasonable response to transforaminal and other epidural injections of steroids, but was left with radicular symptoms and significant pain which was distressing to him. He also exhibited weakness on physical exam in the L45 distribution and sensory deficits in that distribution as well. Patient also had significant sciatic notch tenderness.

Following the foraminoplasty procedure (the intraforaminal procedure described herein), during which the medial and inferiolateral intraforaminal ligaments were distracted or lysed by blunt dissection, and epidural foraminal adhesions were lysed, the patient noted an immediate and profound return of sensory function and had within a few minutes return of full motor strength in his right lower extremity, except for a very mild decrease in right plantar flexion. The patient did well on follow-up and had only mild back stiffness. On follow-up, the patient exhibited normal right lower extremity sensory and motor function. This was confirmed on physical exam.

### Example 8

A middle-aged man suffered from severe lower back pain and radiculopathic symptoms for several years. An EMG was obtained which confirmed significant radiculopathy. Following lysis or distraction of intraforaminal and periforaminal fibrous tissue bands, the patient's symptoms resolved within minutes.

This patient had previously required high dose opioids and other medications to control symptoms. The patient had previously not been able to walk for any significant period of time. Following the procedure, the patient walked for five hours without symptoms. The patient's remaining symptoms appear to be limited to mild to moderate axial back pain. A repeat EMG documented that the previously-document radiculopathy was gone. These results document electrophysiologic evidence that lysing or distracting intraforaminal or periforaminal ligaments, false ligaments, or fibrous or other tissue bands can relieve radiculopathic symptoms and signs, and can even reverse chronic neuropathic changes.

### Example 9

A middle-aged woman presented with radiculopathic pain, numbness, and weakness that had persisted for more than two years. The patient had previously undergone epidural steroid injections with some benefit, but with persistent severe radicular pain, numbness, and weakness. Upon examination, the patient exhibited severe weakness on knee flexion and dorsal plantar retroflexion and weakness in left lower extremity muscles. The patient also exhibited classic radicular distribution of severe numbness. Magnetic resonance imaging results were significant only for a minor bulging intervertebral disc.

The patient underwent the lateral foraminoplasty procedure described herein as periforaminal procedure. The dissecting instrument was directed to a lateral foraminal area at which no intervertebral disc was present. Periforaminal fibrous or ligamentous-type tissue was distracted and likely severed. During the procedure, the corresponding nerve root was contacted by the dissecting instrument, and the normal electrical (i.e., pins and needles) diathesis response was effected. The dissecting instrument was moved within the inferiolateral peliforaminal area, and the patent noticed a profound and rapid return of normal sensation in her left lower limb. Concurrently, radicular pain decreased gradually and disappeared within minutes.

Following the procedure, the patient was able to walk without a limp, whereas she walked with a limp prior to the procedure. The patient reported normality of sensory and motor functions which had not been experienced for more than two years prior to the procedure.

The results shown in this example demonstrate that lysis or distraction of periforaminal fibrous tissues can lead to total or near-total resolution of radiculopathic symptoms and signs.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

What is claimed is:

1. A method of relieving a spinal nerve impingement disorder of a vertebrate, the method comprising displacing at least one of a transforaminal ligament and a periforaminal ligament from a spinal neural structure (SNS) to a degree sufficient to relieve the disorder.

2. The method of claim 1, wherein the ligament and the SNS are adhered prior to displacing the ligament.

3. The method of claim 2, wherein the ligament and the SNS are not adhered after displacing the ligament.

4. The method of claim 1, wherein at least a portion of the ligament is removed from the vertebrate.

5. The method of claim 1, wherein the ligament is displace from the SNS using a percutaneously inserted instrument.

6. The method of claim 5, wherein the instrument is selected from the group consisting of picks, needles, probes, cannulas, elevators, spatulas, spoons, separators, dissectors, hooks, awls, burnishers, rasps, curettes, drills, bits, screws, trephines, scalpels, scissors, forceps, retractors, pliers, syringes, and balloons.

7. The method of claim 5, wherein the ligament is displaced by urging the instrument against the ligament.

8. The method of claim 5, wherein the non-neural tissue is displaced by urging the instrument against the SNS.

9. The method of claim 5, wherein the ligament is displaced by pressure applied by a fluid supplied using the instrument.

10. The method of claim 1, wherein the ligament is displaced by ablating a portion of the ligament.

11. The method of claim 10, wherein the ligament is ablated using an agent selected from the group consisting of heat, cold, electricity, abrasion, ultrasound, vibration, laser light, maser radiation, fluid pressure, gamma radiation, a chemical, and an enzyme.
